(19) United States
(12) Patent Application Publication
(10) Pub. No.: US 2020/0152303 A1
(43) Pub. Date: May 14, 2020

(54) METHODS FOR ELECTRONICALLY PROCESSING INSURANCE CLAIMS AND DEVICES THEREOF

(71) Applicant: Mitchell International, Inc., San Diego, CA (US)

(72) Inventors: Michele Hibbert-Iacobacci, Ramona, CA (US); Valerie Lindgren, San Diego, CA (US); Vicki Dunbar, San Diego, CA (US); Susan Englehart, San Diego, CA (US)

(21) Appl. No.: 16/678,216

(22) Filed: Nov. 8, 2019

Related U.S. Application Data
(60) Provisional application No. 62/758,185, filed on Nov. 9, 2018.

(51) Int. Cl. G16H 15/00

(52) U.S. Cl. CPC G16H 15/00

(57) ABSTRACT

Disclosed technology includes identifying injury data in an electronic medical claim data associated with a claimant based diagnosis code data present in the electronic medical claim data. Presence of historical claim data including initial injury data represented by initial diagnosis code data associated with the claimant for the electronic medical claim data is determined. At least one type of clinical correlation of a plurality of clinical correlations between the identified injury data represented by the diagnosis code data and the initial injury data represented by the initial diagnosis code data is identified when historical claim data is determined to be present for the claimant. Claim processing guidance data to process the electronic medical claim data is provided based on the identified at least one type of correlation between the identified injury data represented by the diagnosis code data and the initial injury data represented by the initial diagnosis code data.

This application claims the benefit of U.S. Provisional Patent Application Ser. No. 62/758,185, filed Nov. 9, 2018, which is hereby incorporated by reference in its entirety.

### FIELD

This technology generally relates to managing insurance claims, and particularly relates to, electronically processing insurance claims.

### BACKGROUND

To assist with managing insurance claims, various types of coding systems have evolved to communicate claim related information in a nationally accepted common format that is readable by electronic claims payment systems. One of these coding systems is the International Classifications of Diseases (ICD) code system which provides codes that identify what service has been provided along with the diagnosis, symptom, complaint, condition or problem as well as the particular resources used.

Unfortunately, existing technological solutions to process these codes when managing insurance claims are typically limited to simply providing these claims with the codes electronically, but provide little further assistance and there is still heavy reliance on manual analysis of the underlying claim and code(s). In particular, these received claims often contain codes developed for different applications and purposes which do not transfer properly when assessing an insurance claim. Additionally, these received claims contain claim related data in addition to these codes which simply on their face fail to provide sufficient information to accurately and consistently identify whether or not one or more identified injuries are related to the insurance claim which has been submitted. This lack of any technological tools to accurately and consistently assess these claims with this uncorrelated data and codes designed for other purposes, results in errors and inconsistencies and thus inappropriate reimbursements.

### SUMMARY

A method for electronically processing insurance claims includes identifying injury data in an electronic medical claim data associated with a claimant based diagnosis code data present in the electronic medical claim data. Presence of historical claim data including initial injury data represented by initial diagnosis code data associated with the claimant for the electronic medical claim data is determined. At least one type of clinical correlation of a plurality of clinical correlations between the identified injury data represented by the diagnosis code data and the initial injury data represented by the initial diagnosis code data is identified when historical claim data is determined to be present for the claimant. Claim processing guidance data to process the electronic medical claim data is provided based on the identified at least one type of correlation between the identified injury data represented by the diagnosis code data and the initial injury data represented by the initial diagnosis code data.

A non-transitory computer readable medium having stored thereon instructions for electronically processing insurance claims comprising executable code, which when executed by at least one processor, causes the processor to identify injury data in an electronic medical claim data associated with a claimant based diagnosis code data present in the electronic medical claim data. Presence of historical claim data including initial injury data represented by initial diagnosis code data associated with the claimant for the electronic medical claim data is determined. At least one type of clinical correlation of a plurality of clinical correlations between the identified injury data represented by the diagnosis code data and the initial injury data represented by the initial diagnosis code data is identified when historical claim data is determined to be present for the claimant. Claim processing guidance data to process the electronic medical claim data is provided based on the identified at least one type of correlation between the identified injury data represented by the diagnosis code data and the initial injury data represented by the initial diagnosis code data.

A claim processing apparatus includes a memory coupled to the processor which is configured to be capable of executing programmed instructions comprising and stored in the memory to identify injury data in an electronic medical claim data associated with a claimant based diagnosis code data present in the electronic medical claim data. Presence of historical claim data including initial injury data represented by initial diagnosis code data associated with the claimant for the electronic medical claim data is determined. At least one type of clinical correlation of a plurality of clinical correlations between the identified injury data represented by the diagnosis code data and the initial injury data represented by the initial diagnosis code data is identified when historical claim data is determined to be present for the claimant. Claim processing guidance data to process the electronic medical claim data is provided based on the identified at least one type of correlation between the identified injury data represented by the diagnosis code data and the initial injury data represented by the initial diagnosis code data.

Accordingly, this technology provides methods, non-transitory computer readable medium, and apparatuses that assist with more accurately and consistently processing claims than would be possible with a manual review. In particular, examples of the claimed technology are able to extract and correlate data and codes enabling enhanced processing of electronic claims to not only assess provide claim data, but to quantify any injury data to identify the relationship between the current injury and a historical injury.

### BRIEF DESCRIPTION OF THE DRAWINGS

FIG. 1 is a block diagram of an environment with an example of a claim processing apparatus that assists with electronically processing insurance claims;

FIG. 2 is a block diagram of the example of the claim processing apparatus shown in FIG. 1;

FIG. 3 is a flowchart of an example of a method for electronically processing insurance claims;

FIG. 4 is an exemplary block diagram medical claim data relating to a nature of an injury, a diagnosis code, and a level of severity of the injury; and

FIG. 5 is an exemplary table illustrating injuries represented by the diagnosis code and the corresponding textual description of the injury.

### DETAILED DESCRIPTION

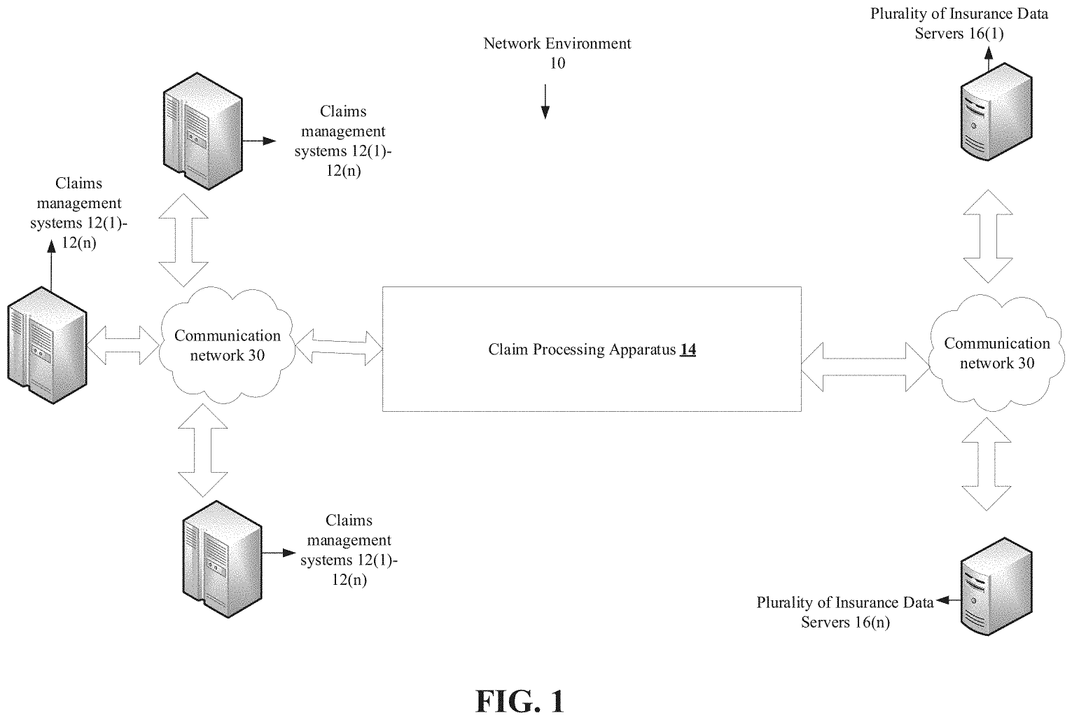

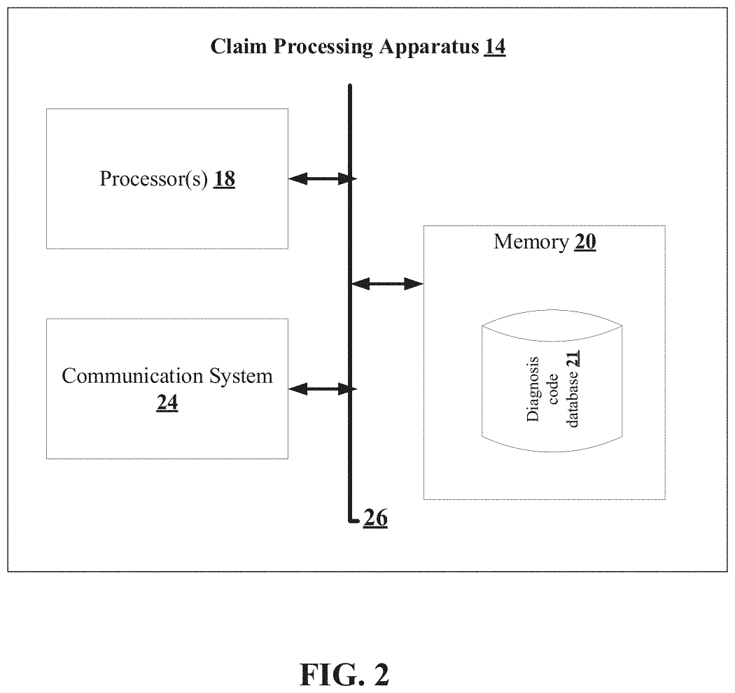

An environment 10 with an example of a claim processing apparatus 14 is illustrated in FIGS. 1-2. In this particular example, the environment 10 includes the claim processing apparatus 14, claims management systems 12(1)-12(n), plurality of insurance data servers 16(1)-16(n) coupled via one or more communication networks 18, although the environment could include other types and numbers of systems, devices, components, and/or other elements as is generally known in the art and will not be illustrated or described herein. This technology provides a number of advantages including providing methods, non-transitory computer readable medium, and apparatuses that electronically processes insurance claims.

Referring more specifically to FIGS. 1-2, the claim processing apparatus 14 is programmed to electronically process insurance claims, although the apparatus can perform other types and/or numbers of functions or other operations and this technology can be utilized with other types of claims. In this particular example, the claim processing apparatus 14 includes a processor 18, a memory 20, and a communication system 24 which are coupled together by a bus 26, although the claim processing apparatus 14 may comprise other types and/or numbers of physical and/or virtual systems, devices, components, and/or other elements in other configurations.

The processor 18 in the claim processing apparatus 14 may execute one or more programmed instructions stored in the memory 20 for processing insurance claims as illustrated and described in the examples herein, although other types and numbers of functions and/or other operations can be performed. The processor 18 in the claim processing apparatus 14 may include one or more central processing units and/or general purpose processors with one or more processing cores, for example.

The memory 20 in the claim processing apparatus 14 stores the programmed instructions and other data for one or more aspects of the present technology as described and illustrated herein, although some or all of the programmed instructions could be stored and executed elsewhere. A variety of different types of memory storage devices, such as a random access memory (RAM) or a read only memory (ROM) in the system or a floppy disk, hard disk, CD ROM, DVD ROM, or other computer readable medium which is read from and written to by a magnetic, optical, or other reading and writing system that is coupled to the processor 18, can be used for the memory 20. Further, as illustrated in FIG. 2, memory 20 includes a diagnosis code database 21 that includes injury data for all the diagnosis code, although the diagnosis code database 21 can include other types or amounts of information. The application(s) can be implemented as module or components of another application. Further, the application(s) can be implemented as operating system extensions, module, plugins, or the like. The application(s) can be implemented as module or components of another application. Further, the application(s) can be implemented as operating system extensions, module, plugins, or the like. Even further, the application(s) may be operative in a cloud-based computing environment. The application(s) can be executed within virtual machine(s) or virtual server(s) that may be managed in a cloud-based computing environment. Also, the application(s), including the claim processing apparatus 14 itself, may be located in virtual server(s) running in a cloud-based computing environment rather than being tied to one or more specific physical network computing devices. Also, the application(s) may be running in one or more virtual machines (VMs) executing on the claim processing apparatus 14.

The communication system 24 in the claim processing apparatus 14 operatively couples and communicates between one or more of the claims management systems 12(1)-12(n) and one or more of the plurality of insurance data servers 16(1)-16(n), which are all coupled together by one or more of the communication networks 30, although other types and numbers of communication networks or systems with other types and numbers of connections and configurations to other devices and elements. By way of example only, the communication networks 30 can use TCP/IP over Ethernet and industry-standard protocols, including NFS, CIFS, SOAP, XML, LDAP, SCSI, and SNMP, although other types and numbers of communication networks, can be used. The communication networks 30 in this example may employ any suitable interface mechanisms and network communication technologies, including, for example, any local area network, any wide area network (e.g., Internet), teletraffic in any suitable form (e.g., voice, modem, and the like), Public Switched Telephone Network (PSTNs), Ethernet-based Packet Data Networks (PDNs), and any combinations thereof and the like.

In this particular example, each of the claims management systems 12(1)-12(n) may submit requests for electronically processing the insurance claims, although the claim processing apparatus 14 can receive other types or requests from the claims management systems 12(1)-12(n). Each of the claims management systems 12(1)-12(n) may include a processor, a memory, user input device, such as a keyboard, mouse, and/or interactive display screen by way of example only, a display device, and a communication interface, which are coupled together by a bus or other link, although each may have other types and/or numbers of other systems, devices, components, and/or other elements.

The plurality of insurance data servers 16(1)-16(n) may store and provide historical insurance claims with initial injury codes and/or other data necessary to electronically process insurance claims, by way of example only, to the claim processing apparatus 14 via one or more of the communication networks 30, for example, although other types and/or numbers of storage media in other configurations could be used. In this particular example, each of the plurality of insurance data servers 16(1)-16(n) may comprise various combinations and types of storage hardware and/or software and represent a system with multiple network server devices in a data storage pool, which may include internal or external networks. Various network processing applications, such as CIFS applications, NFS applications, HTTP Web Network server device applications, and/or FTP applications, may be operating on the plurality of insurance data servers 16(1)-16(n) and may transmit data in response to requests from the claim processing apparatus 14. Each the plurality of insurance data servers 16(1)-16(n) may include a processor, a memory, and a communication interface, which are coupled together by a bus or other link, although each may have other types and/or numbers of other systems, devices, components, and/or other elements.

Thus, the technology disclosed herein is not to be construed as being limited to a single environment and other configurations and architectures are also envisaged. For example, the plurality of claims management systems 12(1)-12(n) depicted in FIG. 1 can operate within claim processing apparatus 14 rather than as a stand-alone server communicating with claim processing apparatus 14 via the communication network(s) 30.

While the claim processing apparatus 14 is illustrated in this example as including a single device, the claim processing apparatus 14 in other examples can include a plurality of devices or blades each with one or more processors each processor with one or more processing cores that implement one or more steps of this technology. In these examples, one or more of the devices can have a dedicated communication interface or memory. Alternatively, one or more of the devices can utilize the memory, communication interface, or other hardware or software components of one or more other communicably coupled of the devices. Additionally, one or more of the devices that together comprise claim processing apparatus 14 in other examples can be standalone devices or integrated with one or more other devices or applications, such as one of the plurality of insurance data servers 16(1)-16(n) or, the claim processing apparatus 14, or applications coupled to the communication network(s), for example. Moreover, one or more of the devices of the claim processing apparatus 14 in these examples can be in a same or a different communication network 30 including one or more public, private, or cloud networks, for example.

Although the exemplary network environment 10 with the claim processing apparatus 14, the claims management systems 12(1)-12(n), the plurality of insurance data servers 16(1)-16(n), and the communication networks 30 are described and illustrated herein, other types and numbers of systems, devices, components, and/or elements in other topologies can be used. It is to be understood that the systems of the examples described herein are for exemplary purposes, as many variations of the specific hardware and software used to implement the examples are possible, as will be appreciated by those skilled in the relevant art(s).

In addition, two or more computing systems or devices can be substituted for any one of the systems or devices in any example. Accordingly, principles and advantages of distributed processing, such as redundancy and replication also can be implemented, as desired, to increase the robustness and performance of the devices, apparatuses, and systems of the examples. The examples may also be implemented on computer system(s) that extend across any suitable network using any suitable interface mechanisms and traffic technologies, including by way of example only teletraffic in any suitable form (e.g., voice and modem), wireless traffic media, wireless traffic networks, cellular traffic networks, G3 traffic networks, Public Switched Telephone Network (PSTNs), Packet Data Networks (PDNs), the Internet, intranets, and combinations thereof.

The examples also may be embodied as a non-transitory computer readable medium having instructions stored thereon for one or more aspects of the present technology as described and illustrated by way of the examples herein, as described herein, which when executed by the processor, cause the processor to carry out the steps necessary to implement the methods of this technology as described and illustrated with the examples herein.

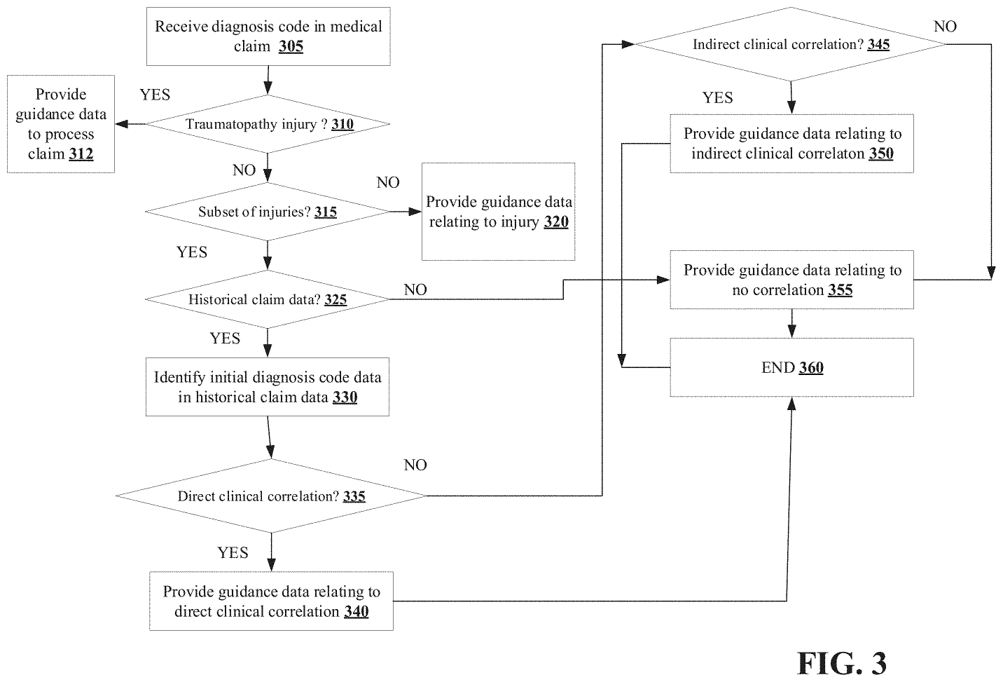

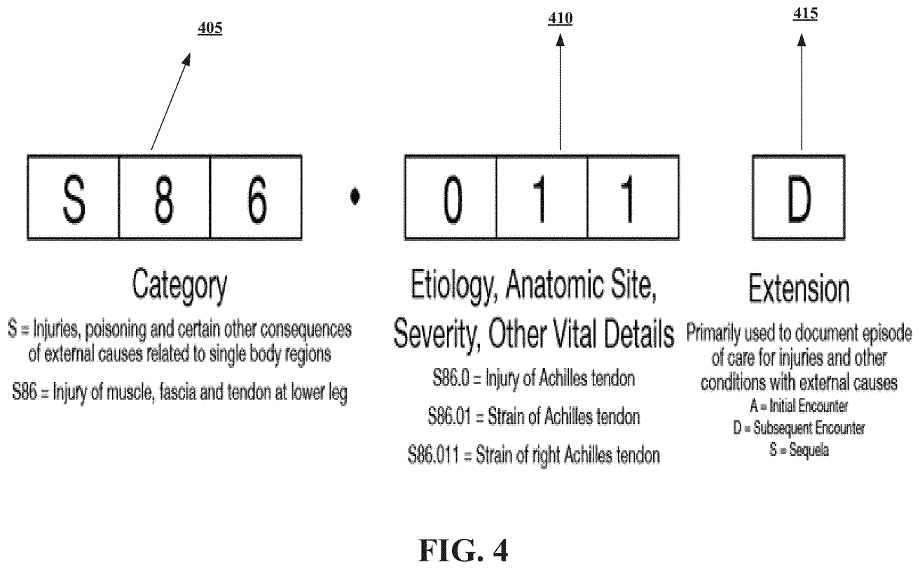

An example of a method for electronically processing insurance claims will now be described with reference to FIGS. 1-5. In particular, referring to FIG. 3 the exemplary method begins at step 305 where the claim processing apparatus 14 receives an electronic medical bill of a patient from one of the plurality of claims management systems 12(1)-12(n), although the claim processing apparatus 14 can receive other types of information from the plurality of claims management systems 12(1)-12(n). In this example, the received medical bill includes claim data associated with the one or more injuries and their corresponding initial diagnosis code along with the injury data indicating the level of severity of the injuries, although the medical bill can include other types or amounts of information. One example of the initial diagnosis code along with the level of severity of the injury is illustrated in FIG. 4, by way of example. By way of example, if the initial diagnosis code is S86.011.D, then the category 405 describes S86 as the injury, poisoning and certain other consequences of external causes related to single body regions. The next three numeric values 410 represents the etiology, anatomic site, severity, and other vital details. By way of example, a lower numeric value can indicate a lower severity level, a higher numeric value can indicate higher severity level and in between the lower numeric value and the higher numeric value can indicate medium severity, although in other examples, lower severity level of the injury can be represented by a higher numeric value and the higher severity level can be represented by a lower numeric value. For purpose of further illustration, if the range of the numeric value is between 0-3, then the severity level could be low; if the range of the numeric value is between 4-6, then the severity level could be medium; and for a range of the numeric value between 7-9, the severity level can be high. Finally, the last alphabet 415 represents the extension that is primarily used to document episode of care for injuries and other conditions with external causes. For example, A indicates that injury is from an initial encounter, D indicates that injury is from a subsequent encounter, and S indicates is sequela.

Upon receiving the electronic medical claim, the claim processing apparatus 14 extracts the diagnosis code from the electronic medical claim. In this example, the electronic medical claim includes a data field with name diagnosis code for each of the injury identified in the electronic claim and the claim processing apparatus 14 extracts the alpha-numeric characters present within the diagnosis code field of the electronic medical claim, although other techniques can be used to identify and extract the diagnosis code from the electronic medical claim. Further, the claim processing apparatus 14 extracts the severity indicator from the extracted diagnosis code. As illustrated above, the severity indicator is the third numeric value from left of the numeric values 410 illustrated in FIG. 4, although other types or amounts of information within the diagnosis code can provide the severity indicator.

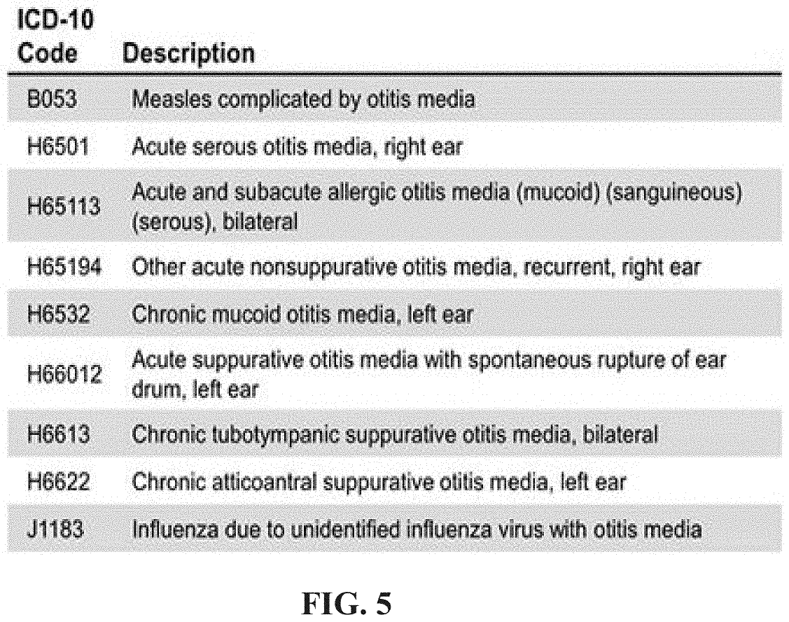

In step 310, the claim processing apparatus 14 determines when the injury in the received electronic medical claim is a traumatopathic injury by correlating at least the first three characters of the extracted diagnosis against the description of the injury stored in a table within the diagnosis code database 21, although the claim processing apparatus 14 can use other techniques to determine when the injury is a traumatopathic injury. In this example, traumatopathic injury relates to pathologic condition resulting from violence or wounds, although traumatopathic injury can include other types or amounts of injuries. By way of example, FIG. 5 represents the diagnosis code and the corresponding description of the injury that is stored within the diagnosis code database 21. Accordingly, when the claim processing apparatus 14 determines that the injury in the received electronic medical claim is a traumatopathic injury, then the Yes branch is taken to step 312.

In step 312, the claim processing apparatus 14 provides the guidance data to process the received electronic medical claim to the requesting one of the plurality of claims management systems 12(1)-12(n), although other types or amounts of information can be provided to the requesting one of the plurality of claims management systems 12(1)-12(n). Optionally, the exemplary flow can proceed to step 325 which will be further illustrated below. In another example, the exemplary flow can end at step 360 after providing the guidance data.

However, back in step 310, when the claim processing apparatus 14 determines that the injury in the received electronic medical claim is not a traumatopathic injury, then the No branch is taken to step 315.

In step 315, the claim processing apparatus 14 determines when the injury in the received electronic medical claim is one of a plurality of injuries by correlating at least the first three characters of the extracted diagnosis against the description of the injury stored in a table within the diagnosis code database 21, although the claim processing apparatus 14 can use other techniques to determine when the types of injuries. By way of example, types of injuries can include extreme injury or traumatic injury. In this example, extreme injury relates to injury that is life threatening, and traumatic injury relates to a physical injury of sudden onset and severity which require immediate medical attention. Accordingly, when the claim processing apparatus 14 determines that the injury in the received electronic medical claim is neither extreme nor traumatic, then the No branch is taken to step 320.

In step 320, the claim processing apparatus 14 provides guidance data to the requesting one of the plurality of claims management systems 12(1)-12(n) indicating that the injury represented by the diagnosis code in the received electronic medical claim is not trumatopathic injury, extreme injury, or traumatic injury, and the exemplary flow can optionally proceed to step 325. In another example, the exemplary flow can proceed to step 360 where the exemplary method ends. In this example, the guidance data can be provided via a graphical user interface to the requesting one of the plurality of claims management systems 12(1)-12(n), although the guidance data can be provided using other techniques.

However, back in step 315, when the claim processing apparatus 14 determines that the injury in the received electronic medical claim is either extreme or traumatic, then the Yes branch is taken to step 325.

In step 325, the claim processing apparatus 14 determines when there is one or more historical medical claim(s) data associated with the claimant from the insurance data servers 16(1)-16(n) using a unique identification number associated with claimant, although the historical medical claim(s) data can be obtained from other memory locations using other techniques. In this example, each claimant is associated with a unique identification number and the claim processing apparatus 14 searches the plurality of insurance data servers 16(1)-16(n) for historical medical claims using the unique identification number, although the claim processing apparatus 14 can use other techniques to identify the historical medical claims. Accordingly, when the claim processing apparatus 14 does not identify any historical medical claim associated with the claimant, then the No branch is taken to step 355 where the exemplary method ends after providing guidance data to the requesting one of the plurality of claims management systems 12(1)-12(n) indicating that no initial diagnosis code is available for the claimant. However, when the claim processing apparatus 14 determines that there is at least one historical medical claim present for the claimant, then the Yes branch is taken to step 330.

In step 330, the claim processing apparatus 14 identifies the initial diagnosis code data present in the obtained historical medical claim(s) by parsing each of the obtained historical medical claim and extracting the initial diagnosis code data present within the initial diagnosis data field, although other techniques can be used to identify the initial diagnosis code.

In step 335, the claim processing apparatus 14 determines when there is a direct clinical correlation between the injury represented by the initial diagnosis code identified in the historical medical claims and the injury represented by the diagnosis code extracted from the received electronic medical claim in step by applying natural language processing technique on the description of the injury associated with the initial diagnosis code and the diagnosis code, although other techniques can be used to identify an direct clinical correlation. In this example, a clinical correlation relates to a process to make a diagnosis on the claimant to treat the injury suffered by the claimant, although clinical correlation can include other types or amounts of information. Additionally, direct clinical correlation relates to a correlation that can be identified without requirement for additional medical data, although correlation can include other types or amounts of information. Further in this example and as illustrated above, the diagnosis code database 21 includes a description of the injury for the diagnosis code extracted from the received electronic claim and similarly, the initial diagnosis code identified in the historical bill also includes a description of the injury that can be obtained either from the diagnosis code database 21 or the plurality of insurance data servers 16(1)-16(n). Further, the claim processing apparatus 14 applies natural language processing technique on both description of the injury associated with the initial diagnosis code and the diagnosis code to determine when there is a direct clinical correlation, although other techniques can be used to determine the direct clinical correlation. By way of example, when claimant's historical claim includes an initial diagnosis code M23.203, which represents derangement of unspecified medial meniscus due to old tear or injury, right knee and the diagnosis code in the extracted from the received electronic claim includes the diagnosis code, S83.211A, which represents bucket-handle tear of medial meniscus with a current injury to the right knee as an initial encounter, then the claim processing apparatus 14 can determine a direct clinical correlation. Accordingly, when the claim processing apparatus 14 determines that there is no direct clinical correlation between the injury represented in the initial diagnosis code and the injury represented in the diagnosis code present in the received electronic medical claim, then the No branch is taken to 345 that will be further illustrated below. However, when the claim processing apparatus 14 determines that there is a direct clinical correlation, then the Yes branch is taken to step 340.

In step 340, the claim processing apparatus 14 provides guidance data to the requesting one of the plurality of claims management systems 12(1)-12(n) indicating that there is a direct clinical correlation between the injury in the received electronic medical claim and the injury present in the historical medical claim. Additionally, the claim processing apparatus 14 may also provide the guidance data to reimburse the amount present in the received electronic claim. In this example, the guidance data can be provided via a graphical user interface to the requesting one of the plurality of claims management systems 12(1)-12(n), although the guidance data can be provided using other techniques. Further, the exemplary flow proceeds to step 360 where the exemplary method ends at step 360.

However, back in step 335, when the claim processing apparatus 14 determines that there is no direct clinical correlation between the injury represented by the initial diagnosis code in the historical medical claim and the injury represented by the diagnosis code in the received electronic claim, the No branch is taken to step 345.

In step 345, the claim processing apparatus 14 determines when there is an indirect clinical correlation between the injury represented by the initial diagnosis code identified in the historical medical claims and the injury represented by the diagnosis code extracted from the received electronic medical claim in step by applying natural language processing technique on the description of the injury associated with the initial diagnosis code and the diagnosis code, although other techniques can be used to identify an indirect clinical correlation. In this example, indirect clinical correlation relates to a correlation that can be identified through additional medical data, although indirect clinical correlation can include other types or amounts of information. By way of example, if the claimant's historical claim includes an initial diagnosis code F43.12, which represents post-traumatic stress disorder and is categorized as chronic, and the received electronic claim includes a diagnosis code S13.130A which represents subluxation of C2/C3 cervical vertebrae during an initial encounter, then the claim processing apparatus 14 determines indirect clinical correlation. Accordingly, when the claim processing apparatus 14 determines that there is an indirect clinical correlation between the injury represented by the initial diagnosis code identified in the historical medical claims and the injury represented by the diagnosis code extracted from the received electronic medical claim, then the Yes branch is taken to step 350.

In step 350, the claim processing apparatus 14 provides guidance data to the requesting one of the plurality of claims management systems 12(1)-12(n) indicating that there is an indirect clinical correlation between the injury represented by the initial diagnosis code identified in the historical medical claims and the injury represented by the diagnosis code extracted from the received electronic medical claim. Additionally, claim processing apparatus 14 may provide guidance data to the requesting one of the plurality of claims management systems 12(1)-12(n) to request additional medical data to confirm the indirect clinical correlation. The exemplary flow ends at step 360.

However, back in step 345, when the claim processing apparatus 14 determines that there is no indirect clinical correlation between the injury represented by the initial diagnosis code identified in the historical medical claims and the injury represented by the diagnosis code extracted from the received electronic medical claim, then the No branch is taken to step 355.

In step 355, the claim processing apparatus 14 provides guidance data to the requesting one of the plurality of claims management systems 12(1)-12(n) indicating that there is no direct clinical correlation or indirect clinical correlation between the between the injury represented by the initial diagnosis code identified in the historical medical claims and the injury represented by the diagnosis code extracted from the received electronic medical claim. Additionally, the claim processing apparatus 14 can also provide guidance data indicating not to reimburse the amount in the received electronic medical claim and the exemplary flow ends at step 360.

Accordingly, this technology provides methods, non-transitory computer readable medium, and apparatuses that assist with more accurately and consistently processing claims than would be possible with a manual review. In particular, examples of the claimed technology are able to extract and correlate data and codes enabling enhanced processing of electronic claims to not only assess provide claim data, but to quantify any injury data to identify the relationship between the current injury and a historical injury.

Having thus described the basic concept of the technology, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the technology. Additionally, the recited order of processing elements or sequences, or the use of numbers, letters, or other designations therefore, is not intended to limit the claimed processes to any order except as may be specified in the claims. Accordingly, the technology is limited only by the following claims and equivalents thereto.

What is claimed is:

1. A method for electronically processing insurance claims, the method comprising:
identifying, by a computing apparatus, injury data in an electronic medical claim data associated with a claimant based diagnosis code data present in the electronic medical claim data;
determining, by the computing apparatus, when there is historical claim data including initial injury data represented by initial diagnosis code data associated with the claimant for the electronic medical claim data;
identifying, by the computing apparatus, when there is at least one type of clinical correlation of a plurality of clinical correlations between the identified injury data represented by the diagnosis code data and the initial injury data represented by the initial diagnosis code data when historical claim data is determined to be present for the claimant; and
providing, by the computing apparatus, claim processing guidance data to process the electronic medical claim data based on the identified at least one type of correlation between the identified injury data represented by the diagnosis code data and the initial injury data represented by the initial diagnosis code data.

2. The method as set forth in claim 1 wherein the identifying further comprises:
identifying, by the computing apparatus, a direct clinical correlation between the identified injury data represented by the diagnosis code data and the initial injury data represented by the initial diagnosis code data by applying a natural language processing technique on injury description data associated with the identified injury data and the initial injury data, wherein the direct clinical correlation is the at least one type of clinical correlation of the plurality of clinical correlations; and
providing, by the computing apparatus, the claim processing guidance data relating to the identified direct clinical correlation between the identified injury data represented by the diagnosis code data and the initial injury data represented by the initial diagnosis code data.

3. The method as set forth in claim 1 wherein the identifying further comprises:
identifying, by the computing apparatus, an indirect clinical correlation between the identified injury data represented by the diagnosis code data and the initial injury data represented by the initial diagnosis code data by applying the natural language processing technique on injury description data associated with the identified injury data and the initial injury data; and
providing, by the computing apparatus, the claim processing guidance data relating to the identified indirect clinical correlation between the identified injury data represented by the diagnosis code data and the initial injury data represented by the initial diagnosis code data.

4. The method as set forth in claim 1 wherein the identifying further comprises:
determining, by the computing apparatus, when there the at least one type of clinical correlation is absent between the the identified injury data represented by the diagnosis code data and the initial injury data represented by the initial diagnosis code data; and
providing, by the computing apparatus, the claim processing guidance data relating to absence of the at least one type of clinical correlation between the between the identified injury data represented by the diagnosis code data and the initial injury data represented by the initial diagnosis code data.

5. The method as set forth in claim 1 further comprising:
identifying, by the computing apparatus, a severity value indicator associated with the identified injury data represented by the diagnosis code data; and
providing, by the computing apparatus, the claim processing guidance data based on the identified severity value associated with the identified injury data represented by the diagnosis code data.

6. The method as set forth in claim 1 further comprising, providing, by the computing apparatus, the claim processing guidance data for the identified injury data represented by the diagnosis code data based on the identified severity value indicator when there is historical claim data associated with the claimant determined to be absent.

7. A non-transitory computer readable medium having stored thereon instructions for electronically processing insurance claims, comprising executable code, which when executed by at least one processor, cause the processor to:
identify injury data in an electronic medical claim data associated with a claimant based diagnosis code data present in the electronic medical claim data;
determine when there is historical claim data including initial injury data represented by initial diagnosis code data associated with the claimant for the electronic medical claim data;
identify when there is at least one type of clinical correlation of a plurality of clinical correlations between the identified injury data represented by the diagnosis code data and the initial injury data represented by the initial diagnosis code data when historical claim data is determined to be present for the claimant; and
provide claim processing guidance data to process the electronic medical claim data based on the identified at least one type of correlation between the identified injury data represented by the diagnosis code data and the initial injury data represented by the initial diagnosis code data.

8. The medium as set forth in claim 7 wherein the identifying further comprises:
identify a direct clinical correlation between the identified injury data represented by the diagnosis code data and the initial injury data represented by the initial diagnosis code data by applying a natural language processing technique on injury description data associated with the identified injury data and the initial injury data, wherein the direct clinical correlation is the at least one type of clinical correlation of the plurality of clinical correlations; and
provide the claim processing guidance data relating to the identified direct clinical correlation between the identified injury data represented by the diagnosis code data and the initial injury data represented by the initial diagnosis code data.

9. The medium as set forth in claim 7 wherein the identifying further comprises:
identify an indirect clinical correlation between the identified injury data represented by the diagnosis code data and the initial injury data represented by the initial diagnosis code data by applying the natural language processing technique on injury description data associated with the identified injury data and the initial injury data; and
provide the claim processing guidance data relating to the identified indirect clinical correlation between the identified injury data represented by the diagnosis code data and the initial injury data represented by the initial diagnosis code data.

10. The medium as set forth in claim 7 wherein the identifying further comprises:
determine when there the at least one type of clinical correlation is absent between the identified injury data represented by the diagnosis code data and the initial injury data represented by the initial diagnosis code data; and
provide the claim processing guidance data relating to absence of the at least one type of clinical correlation between the between the identified injury data represented by the diagnosis code data and the initial injury data represented by the initial diagnosis code data.

11. The medium as set forth in claim 7 further comprising:
identify a severity value indicator associated with the identified injury data represented by the diagnosis code data; and
provide the claim processing guidance data based on the identified severity value associated with the identified injury data represented by the diagnosis code data.

12. The medium as set forth in claim 7 further comprising, providing the claim processing guidance data for the identified injury data represented by the diagnosis code data based on the identified severity value indicator when there is historical claim data associated with the claimant determined to be absent.

13. A claim processing apparatus comprising:
a processor; and
a memory coupled to the processor which is configured to be capable of executing programmed instructions comprising and stored in the memory to:
identify injury data in an electronic medical claim data associated with a claimant based diagnosis code data present in the electronic medical claim data;
determine when there is historical claim data including initial injury data represented by initial diagnosis code data associated with the claimant for the electronic medical claim data;
identify when there is at least one type of clinical correlation of a plurality of clinical correlations between the identified injury data represented by the diagnosis code data and the initial injury data represented by the initial diagnosis code data when historical claim data is determined to be present for the claimant; and
provide claim processing guidance data to process the electronic medical claim data based on the identified at least one type of correlation between the identified injury data represented by the diagnosis code data and the initial injury data represented by the initial diagnosis code data.

14. The apparatus as set forth in claim 13 wherein the processor is further configured to be capable of executing the stored programmed instructions wherein the identifying further comprises:
identify a direct clinical correlation between the identified injury data represented by the diagnosis code data and the initial injury data represented by the initial diagnosis code data by applying a natural language processing technique on injury description data associated with the identified injury data and the initial injury data, wherein the direct clinical correlation is the at least one type of clinical correlation of the plurality of clinical correlations; and
provide the claim processing guidance data relating to the identified direct clinical correlation between the identified injury data represented by the diagnosis code data and the initial injury data represented by the initial diagnosis code data.

15. The apparatus as set forth in claim 13 wherein the processor is further configured to be capable of executing the stored programmed instructions wherein the identifying further comprises:
identify an indirect clinical correlation between the identified injury data represented by the diagnosis code data and the initial injury data represented by the initial diagnosis code data by applying the natural language processing technique on injury description data associated with the identified injury data and the initial injury data; and
provide the claim processing guidance data relating to the identified indirect clinical correlation between the identified injury data represented by the diagnosis code data and the initial injury data represented by the initial diagnosis code data.

16. The apparatus as set forth in claim 13 wherein the processor is further configured to be capable of executing the stored programmed instructions wherein the identifying further comprises:
determine when there the at least one type of clinical correlation is absent between the identified injury data represented by the diagnosis code data and the initial injury data represented by the initial diagnosis code data; and
provide the claim processing guidance data relating to absence of the at least one type of clinical correlation between the between the identified injury data represented by the diagnosis code data and the initial injury data represented by the initial diagnosis code data.

17. The apparatus as set forth in claim 13 wherein the processor is further configured to be capable of executing the stored programmed instructions to:
identify a severity value indicator associated with the identified injury data represented by the diagnosis code data; and
provide the claim processing guidance data based on the identified severity value associated with the identified injury data represented by the diagnosis code data.

18. The apparatus as set forth in claim 13 wherein the processor is further configured to be capable of executing the stored programmed instructions to providing the claim processing guidance data for the identified injury data represented by the diagnosis code data based on the identified severity value indicator when there is historical claim data associated with the claimant determined to be absent.
